(19) United States
(12) Patent Application Publication
(10) Pub. No.: US 2010/0222565 A1
(43) Pub. Date: Sep. 2, 2010

(54) METHOD OF PRODUCING 2'-DEOXY-5-AZACYTIDINE (DECITABINE)

(76) Inventors: Oliver Jungmann, Donaueschingen (DE); Norbert Kraut, Tengen (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/682,293

(22) PCT Filed: Oct. 10, 2008
(86) PCT No.: PCT/EP08/63581

§ 371 Date: Apr. 9, 2010

(51) Int. Cl. C07H 19/12

(52) U.S. Cl. 536/28.3

(57) ABSTRACT

Method of producing 2′-deoxy-5-azacytidine (Decitabine) by providing a compound of formula (I):

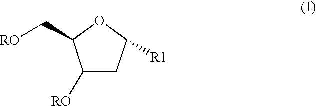

wherein
R is a removable substituent known per se; and R₁ is a removable substituent; further providing a silylated base of formula (II):

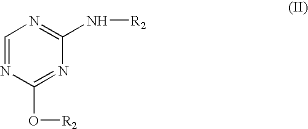

wherein R₂ is a protecting group, preferably a trimethylsilyl TMS)-residue; reacting the compound of formula (I) and the compound of formula (II) together in a suitable anhydrous solvent and in the presence of a suitable catalyst; and removing the substituents R from the compound obtained in order to obtain the compound 2′-deoxy-5-azacytidine (Decitabine), characterized in that said catalyst is selected from the group comprising a salt of an aliphatic sulphonic acid or a salt of a strong inorganic acid.

The present invention refers to a method of producing 2′-deoxy-5-azacytidine (Decitabine) by reacting a glycoside donor preferably a 1-halogen derivative, or an imidate preferably a trichloromethyl derivative, or a thio-alkyl derivative of a blocked monosaccharide with a selected silylated base in the presence of a selected catalyst.

### STATE OF THE ART

Decitabine is a nucleoside and a known pharmaceutically active compound. From U.S. Pat. No. 3,817,980 it is known to synthesize nucleosides by silylating a corresponding nucleoside base and reacting the silylated base with a glycosyl donor preferably a 1-halogen derivative of a blocked monosaccharide in the presence of a selected catalyst. The catalysts used are e.g. selected from SnCl₄, TiCl₄, ZnCl₂, BF₃-etherate, AlCl₃ and SbCl₅. The major disadvantage is that these catalysts are prone to hydrolysis giving irritant hydrolysis products like HCl and/or are forming insoluble oxides (TiO₂, SnO₂), which are difficult to remove from the reaction product. These catalysts are difficult to handle, especially on large scale production.

U.S. Pat. No. 4,082,911 refers to the analogous process of reacting a silylated nucleoside base with a protected derivative of a sugar and proposes to use as catalyst a trialkylsilyl ester of a strong organic acid, such as trimethylsilyl-trifluoromethanesulfonate. U.S. Pat. No. 4,209,613 proposes an improvement for the method disclosed in U.S. Pat. No. 4,082,911 by using a single-step process wherein the trialkylsilyl ester of the strong organic acid, such as trimethylsilyl-trifluoromethanesulfonate, is formed in situ from the free acid by reaction of the free acid with the silylating agent, e.g. trialkylchlorosilane, which is present in the appropriate molar amount. Silylating agents such as trialkylchlorosilane, are very reactive and quickly react to form the trialkylsilyl ester of the free acid present in the reaction mixture.

### DESCRIPTION OF THE INVENTION

It has now been found that a 1-halo monosaccharide derivative can be reacted with a silylated or alkylated 5-azacytosine in the presence of a salt as a catalyst wherein said catalyst is selected from the group comprising a salt of an aliphatic sulphonic acid such as trifluoromethane sulfonate, or a salt of a strong inorganic acid such as a perchlorate. There is no need to use an ester compound as a catalyst. This very much simplifies the production of 2′-deoxy-5-azacytidine (Decitabine) as described in the present invention. Furthermore, using the catalyst of the present invention an improved selectivity in favor of the beta-isomer (β-isomer) may be obtained, e.g. a selectivity of at least 1:2. The reaction of the present invention can be carried out so that about three quarters of the reaction yield is the beta isomer and, depending on the particular reaction conditions, a ratio of the alpha to the beta isomer of 12:88 was obtained. Further, according to the present invention a reaction yield that is higher than 95%, and regularly is within the range of 97-99%, calculated to the total amount of anomers present in the final crude reaction mixture, can be obtained.

The type of catalyst as used according to the present invention is stable under aqueous conditions, easy to handle, does not produce irritant hydrolysis products, and can be easily removed. Additionally, the selectivity of the reaction for obtaining the desired anomer, i.e. the ratio of the alpha/beta anomers, and the final yields are considerably improved.

The present invention is defined in the claims. The present invention refers to a method of producing 2′-deoxy-5-azacytidine (Decitabine) by providing a compound (a blocked monosaccharide derivative) of formula (I):

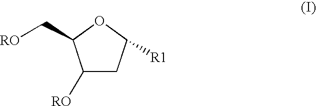

wherein
R is a removable substituent (protecting group) known per se, preferably (C₁-C₈)alkylcarbonyl, or optionally substituted phenylcarbonyl, or optionally substituted benzylcarbonyl;
R₁ is a removable substituent preferably halogen, preferably chlorine, bromine, fluorine, preferably chlorine, or an imidate, preferably trichloromethyl imidate, or a thio-alkyl derivative, preferably —S-methyl;
further providing a silylated base of formula (II):

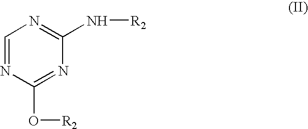

wherein R₂ is a protecting group, preferably a trimethylsilyl (TMS)-residue;
reacting the compound of formula (I) and the compound of formula (II) together in a suitable anhydrous solvent and in the presence of a suitable catalyst, whereby the compound of formula (III):

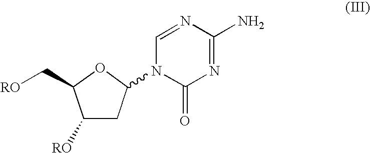

is obtained; and removing the substituent R in order to obtain the compound 2′-deoxy-5-azacytidine (Decitabine), characterized in that said catalyst is selected from the group comprising a salt of an aliphatic sulphonic acid or a salt of a strong inorganic acid.

The present invention refers also to the production of the compound of formula (III) using a catalyst of the present invention, yielding a desired selectivity, preferably in favor of the beta-isomer (β-isomer), preferably at a ratio of at least 1:2, and preferably wherein about three quarters of the reaction yield is the beta isomer. Preferred is the beta-glycoside of formula (III).

If the catalyst used in said reaction is a salt of an aliphatic sulphonic acid, said catalyst preferably is a salt of methylsulphonic acid (mesylate) or of ethylsulphonic acid, or is a salt of a fluorinated aliphatic sulfonic acid, such as a salt of trifluoromethane-sulfonic acid, of pentafluoroethyl-sulfonic acid, or of heptafluoropropyl-sulfonic acid.

If the catalyst used in said reaction is a salt of a strong inorganic acid, said catalyst is a salt composed of an cation as defined herein for the salts of a strong inorganic acid and a non-nucleophilic anion. Said non-nucleophilic anion does not form a complex with said cation in solution. Preferably said salt of a strong inorganic acid is selected from the group comprising: MBPh₄, MB(Me)₄, MPF₆, MBF₄, MClO₄, MBrO₄, MJO₄, M₂SO₄, MNO₃, and M₃PO₄. (M=metal cation; F=fluorine; Cl=chlorine; Br=bromine; B=boron; Ph=phenyl; Me=methyl; P=phosphorous; J=iodine). Preferred are MBPh₄, MB(Me)₄, MPF₆, MBF₄, MClO₄, MBrO₄, MJO₄, most preferred are the salts of perchloric acid (MClO₄) and of tetrafloroboric acid (MBF₄). Most preferred are the salts wherein M=lithium.

Preferred of these salts are the salts of methylsulphonic acid (mesylate), the salts of trifluoromethanesulfonic acid, and the salts of perchloric acid.

Preferred aliphatic sulphonic acid salts, fluorinated aliphatic sulfonic acid salts and salts of a strong inorganic acid are the alkali salts and earth alkali salts, preferably the salts of lithium, sodium, potassium, or magnesium. Preferred are the lithium salts, preferably lithium methylsulphonic acid (lithium mesylate), lithium-trifluoromethanesulfonate (LiOTf, lithium-triflate), lithium perchlorate, and lithium tetrafluoroborate. Also other salts, for example the salts of scandium, such as Sc(OTf)₃, of zinc such as Zn(OTf)₂, or of copper such as Cu(OTf)₂ can be used. However, the lithium salt and especially LiOTf is preferred.

Preferred solvents to carry out the reaction according to the present invention are organic solvents such as benzene, toluene, xylol, or chlorinated solvents, for example dichloromethane, dichloroethane, chloroform, chlorobenzene, or acetonitril and/or propylene carbonate and/or related solvents. Preferred are toluene and chlorinated solvents. Preferred is the use of lithium-trifluoromethanesulfonate (LiOTf) in a chlorinated solvent, preferably in dichloromethane, dichloroethane, chloroform, chlorobenzene and/or in an aromatic solvent like toluene or xylene. Each solvent or mixture of solvents may yield a different selectivity with respect to the beta-isomer (β-isomer). It is no problem for the expert in the art to optimize the catalyst and/or solvent or the mixture of solvents in order to obtain the desired selectivity in favor of the beta-isomer.

The compound of formula (I) is a glycoside donor compound. The preparation of the compound of formula (I) is known per se.

The removable substituent R is preferably (C₁-C₄)alkylcarbonyl, or optionally substituted phenylcarbonyl, like phenylcarbonyl, tolylcarbonyl, xylylcarbonyl or benzylcarbonyl; preferably acetyl or p-chloro-phenylcarbonyl.

The removable substituent R₁ is preferably halogen, preferably chlorine, bromine, fluorine, preferably chlorine, or an imidate, preferably trichloromethyl imidate [—NH—(O)C—CCl₃], or a thioalkyl derivative, preferably —S-methyl.

The compound of formula (II) and its preparation are known. The compound is preferably prepared by reaction of the free base with trimethylchlorosilane or with hexamethyldisilazane.

When reacting the compounds of formulae (I) and (II) together, the reaction temperature generally is within the range of 0° C. to about 90° C., preferably at about room temperature, whereby the components are reacted in about equimolar amounts or with an excess of compound of formula (II). The catalyst is used preferably in a concentration of about 10 mol-% to 100 mol-%, calculated to the total molar presence of the two reacting components. For the expert in the art it is no problem to optimize the molar ratios of the components.

For removing the substituents R from the compound of formula (III) in order to obtain the compound 2′-deoxy-5-azacytidine (Decitabine), containing free hydroxyl groups, known methods are used. The substituents R may be preferably removed, for example, by treatment in an alcoholic solution of ammonia or alcoholates; but other known methods may be applied. The following example illustrates the invention.

### Example 1

(A) A mixture of 5-azacytosine (20 g, 178.4 mmol), ammonium sulfate (2.4 g, 18.16 mmol), and hexamethyldisilazane (160 g, 991.3 mmol) was heated to reflux until a clear solution was obtained. The excess of hexamethyldisilazane was removed in the vacuum at 60° C.

(B) 264 g of dichloromethane, followed by lithium trifluoromethane sulfonate (27.84 g, 178.4 mmol) and the “chloro sugar” C-137: 1-chloro-3,5-di-o-p-chlorobenzoyl-2-deoxy-α-D-ribofuranose [76.67 g, 178.4 mmol, corresponding to compound of formula (I)] were added to the residue obtained in step (A).

(C) The mixture was stirred for 4 hours at ambient temperature (20-25° C.). Reaction yield combined anomers 99.2%, selectivity alpha/beta 27/73.

(D) Then the solvent was removed at 40° C. in the vacuum and the obtained residue was dissolved in 60 g ethyl acetate. The solution was added dropwise to a mixture of 220 g of aqueous sodium hydrogen carbonate (2.5% w-solution), 174 g ethyl acetate, 36 g cyclohexane and 70 g acetonitrile at 30° C. and the obtained reaction mixture is cooled to 0° C. and stirred for 3 hours (h). The precipitate of the blocked (protected) aminotriazine was filtered off, washed with water and finally with a mixture of acetonitrile and ethyl acetate (1:1).

Total yield 79.2 g (87.8%) combined anomers; ratio alpha/beta 31:69. Scheme 1 shows the chemical reaction.

### Example 2

The compound corresponding to formula (III) as obtained in Example 1 is further treated with in an alcoholic solution of ammonia in a known manner so that 2′-deoxy-5-azacytidine (Decitabine) is obtained in practically quantitative yield.

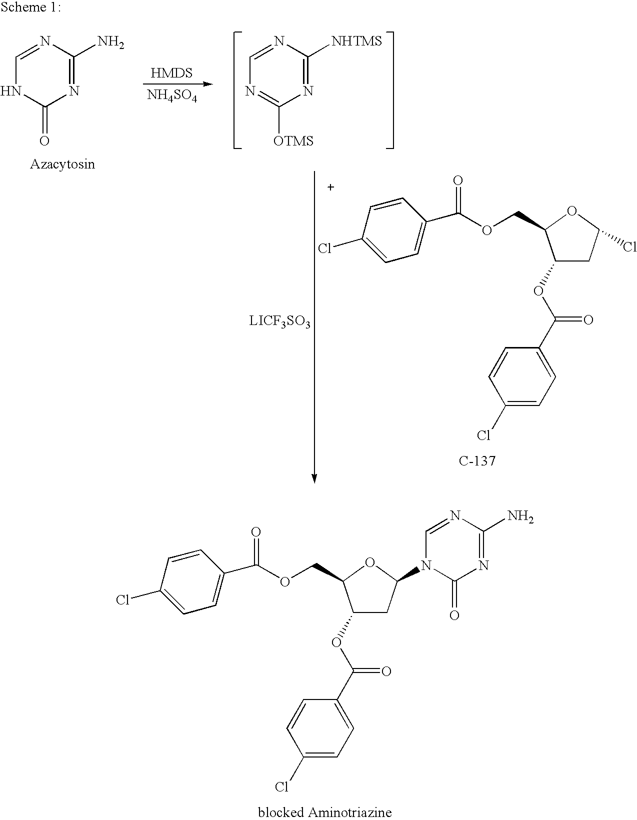

### Example 3

Example 1 was repeated using 1.0 equivalents of lithium mesylate instead of lithium trifluoromethane sulfonate. Reaction yield after step (C): Combined anomers 95.2%, selectivity alpha/beta 60:40.

Total yield after work-up step (D) 85.2% combined anomers; ratio alpha/beta 63:37.

### Example 4

Example 1 was repeated using 1.0 equivalents of lithium perchlorate instead of lithium trifluoromethane sulfonate.

Reaction yield after step (C): Combined anomers 99.4%, selectivity alpha/beta 37:63.

Total yield after work-up step (D) 85.2% combined anomers; ratio alpha/beta 36:64.

### Example 5

Example 1 was repeated using 1.0 equivalents of lithium tetrafluoroborate instead of lithium trifluoromethane sulfonate.

Reaction yield after step (C): Combined anomers 94.5%, selectivity alpha/beta 59:41.

Total yield after work-up step (D) 47.9% combined anomers; ratio alpha/beta 70:30.

### Example 6

Example 1 was repeated using 1.0 equivalents of sodium trifluoromethane sulfonate instead of lithium trifluoromethane sulfonate.

Reaction yield after step (C): Combined anomers 99.2%, selectivity alpha/beta 40:60.

Total yield after work-up step (D) 80.7% combined anomers; ratio alpha/beta 40:60.

### Example 7

Example 1 was repeated using 1.0 equivalents of potassium trifluoromethane sulfonate instead of lithium trifluoromethane sulfonate.

Reaction yield after step (C): Combined anomers 99.0%, selectivity alpha/beta 44:56.

Total yield after work-up step (D) 79.9% combined anomers; ratio alpha/beta 46:54.

### Example 8

Example 1 was repeated [except for step (D)] using 1.0 equivalent of zinc trifluoromethane sulfonate instead of lithium trifluoromethane sulfonate.

Reaction yield after step (C): Combined anomers 96.0%, selectivity alpha/beta 54:46.

### Example 9

Example 1 was repeated using the same volume of toluene instead of dichloromethane as solvent.

Reaction yield after step (C): Combined anomers 99.4%, selectivity alpha/beta 27:73.

Total yield after work-up step (D) 88.7% combined anomers; ratio alpha/beta 31:69.

### Example 10

Example 1 was repeated using the same volume of acetonitrile instead of dichloromethane as solvent.

Reaction yield after step (C): Combined anomers 99.2%, selectivity alpha/beta 50:50.

Total yield after work-up step (D) 82.5% combined anomers; ratio alpha/beta 52:48.

### Example 11

(A) A mixture of 5-azacytosine (0.5 g, 4.46 mmol, 1 equ.), ammonium sulfate (40 mg, 0.3 mmol, 0.07 equ.), and hexamethyldisilazane (4 g, 24.8 mmol, 5.6 equ.) was heated to reflux until a clear solution was obtained. The excess of hexamethyldisilazane was removed in the vacuum at 60° C.

(B) Afterwards 10 ml of dichloromethane, lithium trifluoromethane-sulfonate (0.33 g, 2.11 mmol; 0.47 equ.) and the “chloro sugar” C-137: 1-Chloro-3,5-di-O-p-chlorobenzoyl-2-deoxy-alpha-D-ribofuranose [0.73 g, 1.70 mmol, 0.38 equ.; corresponding to compound of formula (I)] were added to the residue obtained in step (A). The mixture was stirred for 4 hours at ambient temperature (20-25° C.)

Reaction yield combined anomers 99.1%; alpha/beta=16/84.

### Example 12

Example 11 was repeated using 0.47 equivalents of copper trifluoromethane sulfonate instead of lithium trifluoromethane sulfonate.

Reaction yield after step (B): Combined anomers 98.0%, selectivity alpha/beta 42:58.

### Example 13

Example 11 was repeated using 0.47 equivalents of scandium trifluoromethane sulfonate instead of lithium trifluoromethane sulfonate.

Reaction yield after step (B): Combined anomers 88.0%, selectivity alpha/beta 43:57.

### Example 14

Example 11 was repeated using 0.47 equivalents of magnesium trifluoromethane sulfonate instead of lithium trifluoromethane sulfonate.

Reaction yield after step (B): Combined anomers 89.0%, selectivity alpha/beta 58:42.

### Example 15

Example 11 was repeated using the same volume of acetonitrile instead of dichloromethane as solvent.

Reaction yield after step (B): Combined anomers 97.6%, selectivity alpha/beta 39:61.

### Example 16

Example 11 was repeated using the same volume of chlorobenzene instead of dichloromethane as solvent.

Reaction yield after step (B): Combined anomers 96.2%, selectivity alpha/beta 26:74.

### Example 17

Example 11 was repeated using the same volume of propylencarbonate instead of dichloromethane as solvent.

Reaction yield after step (B): Combined anomers 96.8%, selectivity alpha/beta 42:58.

### Example 18

Example 11 was repeated a mixture of 10 ml of dichloromethane and 3.5 ml of xylene instead of 10 ml of pure dichloromethane as solvent.

Reaction yield after step (B): Combined anomers 93.3%, selectivity alpha/beta 27:73.

### Example 19

A mixture of 5-azacytosine (0.5 g, 4.46 mmol, 1 equ.), ammonium sulfate (40 mg, 0.3 mmol, 0.07 equ.), and hexamethyldisilazane (4 g, 24.8 mmol, 5.6 equ.) was heated to reflux until a clear solution was obtained.

(B) Afterwards 10 ml of 1,2-dichlorobenzene, lithium trifluoromethane-sulfonate (0.33 g, 2.11 mmol; 0.47 equ.) and the “chloro sugar” C-137: 1-Chloro-3,5-di-O-p-chlorobenzoyl-2-deoxy-alpha-D-ribofuranose; [1.15 g, 2.68 mmol, 0.60 equ.; corresponding to compound of formula (I)] were added to the residue obtained in step (A). The mixture was stirred for 4 hours at ambient temperature (20-25° C.)

Reaction yield combined anomers 91.2%; alpha/beta=27/73.

### Example 20

Example 19 was repeated using the same volume of 1,2-dichloroethane instead of 1,2-dichlorobenzene as solvent.

Reaction yield after step (B): Combined anomers 93.4%, selectivity alpha/beta 27:73.

### Example 21

(A) A mixture of 5-azacytosine (0.5 g, 4.46 mmol, 1 equ.), ammonium sulfate (40 mg, 0.3 mmol, 0.07 equ.), and hexamethyldisilazane (4 g, 24.8 mmol, 5.6 equ.) was heated to reflux until a clear solution was obtained. The excess of hexamethyldisilazane was removed in the vacuum at 60° C.

(B) Afterwards 10 ml of dichloromethane, lithium trifluoromethanesulfonate (0.33 g, 2.11 mmol; 0.47 equ.) and the “chloro sugar” C-137: 1-Chloro-3,5-di-O-p-chlorobenzoyl-2-deoxy-alpha-D-ribofuranose; [0.38 g, 0.88 mmol, 0.20 equ.; corresponding to compound of formula (I)] were added to the residue obtained in step (A). The mixture was stirred for 4 hours at ambient temperature (20-25° C.)

Reaction yield combined anomers 99.3%; alpha/beta=12/88.

1-15. (canceled)

16. A method of producing 2′-deoxy-5-azacytidine (Decitabine), said method comprising the steps of providing a compound of formula (I): 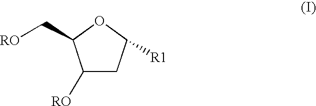
wherein
R is a removable substituent selected from the group consisting of (C₁-C₈)alkylcarbonyl, or optionally substituted phenylcarbonyl, or optionally substituted benzylcarbonyl;
R₁ is a removable substituent selected from the group consisting of halogen, an imidate or a thio-alkyl derivative;
further providing a silylated base of formula (II): 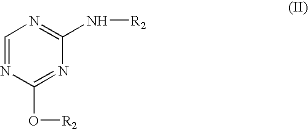
wherein R₂ is a protecting group;
reacting the compound of formula (I) and the compound of formula (II) together in a suitable anhydrous solvent and in the presence of a suitable catalyst, whereby the compound of formula (III): 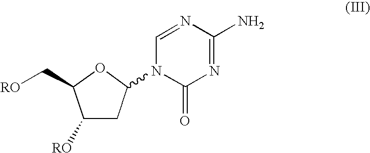
is obtained; and removing the substituents R in order to obtain the compound 2′-deoxy-5-azacytidine (Decitabine), wherein said catalyst is selected from the group comprising a salt of an aliphatic sulphonic acid or a salt of a strong inorganic acid.

17. A method of producing a compound of formula (III): 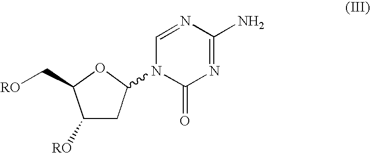
said method comprising the steps of providing a compound of formula (I): 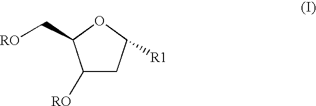
wherein
R is a removable substituent selected from the group consisting of (C₁-C₈)alkylcarbonyl, or optionally substituted phenylcarbonyl, or optionally substituted benzylcarbonyl;
R₁ is a removable substituent selected from the group consisting of halogen, an imidate or a thio-alkyl derivative;
further providing a silylated base of formula (II): 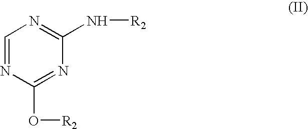
wherein R₂ is a protecting group;
reacting the compound of formula (I) and the compound of formula (II) together in a suitable anhydrous solvent and in the presence of a suitable catalyst, whereby the compound of formula (III): 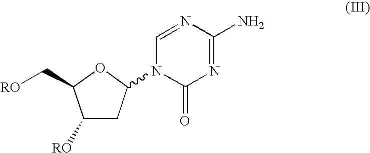
is obtained, wherein said catalyst is selected from the group comprising a salt of an aliphatic sulphonic acid or a salt of a strong inorganic acid.

18. The method according to claim 16, wherein R₁ is a selected from the group consisting of chlorine, bromine, fluorine, chlorine, trichloromethyl imidate, or a thio-alkyl derivative that is —S-methyl.

19. The method according to claim 16, wherein R₂ is a trimethylsilyl (TMS)-residue.

20. The method according to claim 16, wherein R₁ is a selected from the group consisting of chlorine, bromine, fluorine, chlorine, trichloromethyl imidate, or a thio-alkyl derivative that is —S-methyl and R₂ is a trimethylsilyl (TMS)-residue.

21. The method according to claim 16, wherein R is (C₁-C₄)alkylcarbonyl, or optionally substituted phenylcarbonyl or benzylcarbonyl,

22. The method according to claim 16, wherein R is phenylcarbonyl, tolylcarbonyl, xylylcarbonyl, or acetyl or p-chloro-phenylcarbonyl.

23. The method according to claim 17, wherein R₁ is a selected from the group consisting of chlorine, bromine, fluorine, chlorine, trichloromethyl imidate, or a thio-alkyl derivative that is —S-methyl.

24. The method of according to claim 17, wherein R₂ is a trimethylsilyl (TMS)-residue.

25. The method according to claim 17, wherein R₁ is a selected from the group consisting of chlorine, bromine, fluorine, chlorine, trichloromethyl imidate, or a thio-alkyl derivative that is —S-methyl and R₂ is a trimethylsilyl (TMS)-residue.

26. The method according to claim 17, wherein R is (C₁-C₄)alkylcarbonyl, or optionally substituted phenylcarbonyl or benzylcarbonyl,

27. The method according to claim 17, wherein R is phenylcarbonyl, tolylcarbonyl, xylylcarbonyl, or acetyl or p-chloro-phenylcarbonyl.

28. The method according to any one of claims 16-27, wherein the catalyst used in said reaction is a salt of an aliphatic sulphonic acid, or a salt of a fluorinated aliphatic sulfonic acid.

29. The method according to claim 28, wherein the catalyst used in said reaction is a salt of methylsulphonic acid or a salt of ethylsulphonic acid.

30. The method according to claim 28, wherein the catalyst used in said reaction is a salt of trifluoromethane-sulfonic acid, a salt of pentafluoroethyl-sulfonic acid, or a salt of heptafluoropropyl-sulfonic acid.

31. The method according to any one of claims 16-27, wherein the catalyst is an alkali salt or an earth alkali salt.

32. The method of claim 31, wherein the catalyst is a salt of lithium, a salt of sodium, a salt of potassium, or a salt of magnesium.

33. The method of claim 31, wherein the catalyst is lithium methylsulphonic acid and/or lithium-trifluoromethanesulfonate.

34. The method of claim 31, wherein the catalyst is chosen from the salts comprising salts of scandium, of zinc or of copper.

35. The method of claim 31, wherein the catalyst is Sc(OTf)₃, Zn(OTf)₂, or Cu(OTf)₂.

36. The method according to any one of claims 16-27, wherein the catalyst is a salt of a strong inorganic acid composed of an cation and a non-nucleophilic anion which does not form a complex with said cation in solution.

37. The method of claim 36, wherein the catalyst is selected from the group comprising: MBPh₄, MB(Me)₄, MPF₆, MBF₄, MClO₄, MBrO₄, MJO₄, M₂SO₄, MNO₃, and M₃PO₄.

38. The method of claim 36, wherein the catalyst is a salt of perchloric acid and/or a salt of tetrafloroboric acid.

39. The method according to any one of claims 16-27, wherein the solvent to carry out the reaction is chosen from the group comprising organic solvents or chlorinated solvents, xylol, or acetonitril, propylene carbonate.

40. The method of claim 39, wherein the solvent is benzene, toluene or xylene.

41. The method of claim 39, wherein the solvent is dichloromethane, dichloroethane, chloroform, or chlorobenzene.

42. The method according to any one of claims 16-27, wherein the catalyst is lithium-trifluoromethanesulfonate and the solvent is chosen from toluene, xylene, dichloromethane, dichloroethane, chloroform or chlorobenzene.
